(19) United States
(12) Patent Application Publication
(10) Pub. No.: US 2026/0256922 A1
(43) Pub. Date: Sep. 3, 2026

(54) PRODRUGS USEFUL FOR REDUCING ANTHRACYCLINE CARDIOTOXICITY

(71) Applicant: WAKE FOREST UNIVERSITY HEALTH SCIENCES, Winston-Salem, NC (US)

(72) Inventors: John C. LUKESH, III, Clemmons, NC (US); Leslie B. POOLE, Winston-Salem, NC (US)

(21) Appl. No.: 18/864,590

(22) PCT Filed: May 11, 2023
(86) PCT No.: PCT/US2023/066871

§ 371 Date: Nov. 11, 2024

Related U.S. Application Data
(60) Provisional application No. 63/364,616, filed on May 12, 2022.

(51) Int. Cl. A61K 47/54; A61P 35/00

(52) U.S. Cl. CPC A61K 47/549; A61P 35/00

(57) ABSTRACT

Provided herein is a prodrug compound of Formula I, or a pharmaceutically acceptable salt thereof, as well as compositions comprising the compound. Use of the compound or composition for the treatment of cancer is also provided.

### CROSS-REFERENCE TO RELATED APPLICATIONS

This application claims the benefit of U.S. Provisional Application Ser. No. 63/364,616, filed May 12, 2022, the contents of which are incorporated by reference herein.

### STATEMENT OF GOVERNMENT SUPPORT

This invention was made with government support under grant number 2143826 awarded by the National Science Foundation. The government has certain rights in the invention.

### BACKGROUND

Doxorubicin (DOX) is one of the most effective anthracycline anticancer agents in clinical oncology. Its continued use, however, is severely limited by its dose-dependent cardiotoxicity, which stems, in part, from its overproduction of reactive oxygen species (ROS) and often manifests itself as full-blown cardiomyopathy in patients, years after the cessation of treatment. Therefore, identifying DOX analogs, or prodrugs, with a diminished cardiotoxic profile is highly desirable.

Ye et al., Adv. Mater. 29, 702342 (2017) and Yi et al., J. Nanobiotechnol. 19, 134 (2021) describe a ROS responsive DOX prodrug within a nanoparticle or liposomes, respectively. Alov et al., Front. Pharmacol. 13:831791 (March 2022) describe a hydrogen-sulfide-releasing doxorubicin.

However, additional prodrug moieties that may reduce cardiotoxicity are needed.

### SUMMARY

Provided herein according to some embodiments is compound of Formula I:

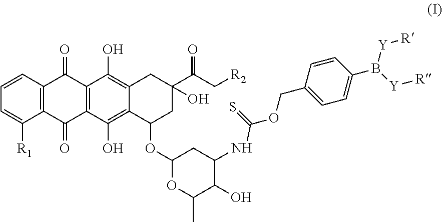

wherein:
R₁ is H or alkoxy (e.g., methoxy);
R₂ is H or hydroxy
each Y is independently NRₐ or O; and
R′, R″, and Rₐ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, hydroxy, alkoxy, carbonyl and/or carboxy groups,
or a pharmaceutically acceptable salt thereof.

In some embodiments, R′ and R″ together with the Y atoms to which they are attached form a boronate or diaminoboryl group.

In some embodiments, the compound is a compound of Formula Ia:

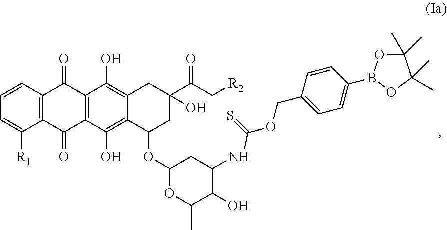

wherein R₁ is H or methoxy, and R₂ is H or hydroxy,
or a pharmaceutically acceptable salt thereof.

In some embodiments, the compound is:

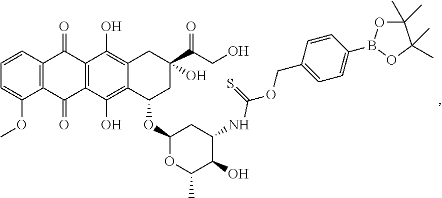 [SMILES: COc1cccc2c1C(=O)c1c(O)c3c(c(O)c1C2=O)C[C@@](O)(C(=O)CO)C[C@@H]3O[C@H]1C[C@H](NC(=S)OCc2ccc(B3OC(C)(C)C(C)(C)O3)cc2)[C@@H](O)[C@H](C)O1]

or a pharmaceutically acceptable salt thereof.

In some embodiments, the compound is:

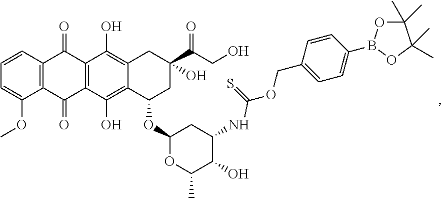 [SMILES: COc1cccc2c1C(=O)c1c(O)c3c(c(O)c1C2=O)C[C@@](O)(C(=O)CO)C[C@@H]3O[C@H]1C[C@H](NC(=S)OCc2ccc(B3OC(C)(C)C(C)(C)O3)cc2)[C@H](O)[C@H](C)O1]

or a pharmaceutically acceptable salt thereof.

In some embodiments, the compound is:

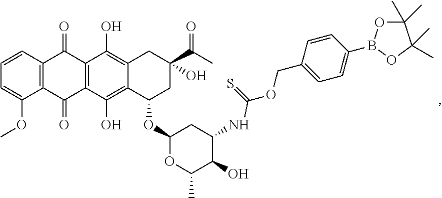 [SMILES: COc1cccc2c1C(=O)c1c(O)c3c(c(O)c1C2=O)C[C@@](O)(C(C)=O)C[C@@H]3O[C@H]1C[C@H](NC(=S)OCc2ccc(B3OC(C)(C)C(C)(C)O3)cc2)[C@@H](O)[C@H](C)O1]

or a pharmaceutically acceptable salt thereof.

In some embodiments, the compound is:

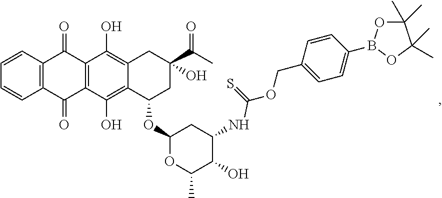 [SMILES: CC(=O)[C@]1(O)Cc2c(O)c3c(c(O)c2[C@@H](O[C@H]2C[C@H](NC(=S)OCc4ccc(B5OC(C)(C)C(C)(C)O5)cc4)[C@H](O)[C@H](C)O2)C1)C(=O)c1ccccc1C3=O]

or a pharmaceutically acceptable salt thereof.

Also provided is a composition comprising a compound as taught herein and a pharmaceutically acceptable carrier.

In some embodiments, the compound is provided as a liposome formulation.

In some embodiments, the compound is provided as a nanoparticle formulation.

Further provided is a method of treating cancer in a subject in need thereof, comprising administering a compound or composition as taught herein to said subject in a treatment effective amount. Also provided is a compound or composition as taught herein for use in the treatment of cancer. Still further provided is the use of a compound or composition as taught herein in the manufacture of a medicament for the treatment of cancer.

In some embodiments, the cancer comprises a solid tumor. In some embodiments, the cancer is a soft tissue carcinoma. In some embodiments, the cancer is breast cancer. In some embodiments, the cancer is a leukemia (e.g., acute lymphocytic leukemia). In some embodiments, the cancer is a lymphoma (e.g., Hodgkin lymphoma or non-Hodgkin lymphoma). In some embodiments, the cancer is bladder cancer. In some embodiments, the cancer is kidney cancer. In some embodiments, the cancer is ovarian cancer.

In some embodiments, the administering is carried out by parenteral administration. In some embodiments, the parenteral administration is intravenous administration.

### BRIEF DESCRIPTION OF THE DRAWINGS

FIG. 1. Time-course for DOX release from prodrugs (10 μM) in ammonium bicarbonate buffer (0.1 M, pH 7.4) at room temperature and in the presence of H₂O₂ (10 μM). A calibration curve was used to determine the concentration of free DOX at each time point by LCMS. Plotted as the mean±standard error of the mean (SEM) from three independent experiments.

FIG. 2. Percentage of released DOX from c1 in response to various biological analytes during an 80 min incubation period at room temperature: (1) ammonium bicarbonate buffer (0.1 M, pH 7.5), (2) 10 μM H₂O₂, (3) 100 μM cysteine, (4) 100 μM homocysteine, (5) 1 mM glutathione, (6) 10 μM glutathione disulfide, (7) 10 UM sodium nitrite, (8) 10 μM sodium hypochlorite, (9) 10 μM superoxide, (10) 10 μM peroxynitrite. A calibration curve was used to determine the concentration of free DOX in response to each analyte. Plotted as the mean±SEM from three independent experiments.

FIG. 3. Methylene blue assay depicting the time-dependent release of H₂S from c1 (40 μM) while in the presence of H₂O₂ (40 μM) and carbonic anhydrase (CA). Plotted as the mean±SEM from three independent experiments. Data were collected in the presence (circles) or absence (squares) of H₂O₂.

FIG. 4. Uptake of DOX and prodrugs c1 and c2 by H9C2 cardiomyoblasts. H₉C2 cells cultures grown overnight on chambered coverslips in media with 10% serum were switched to Fluorobrite DMEM imaging media supplemented with 5% serum and prepared for live-cell imaging on a Zeiss LSM 880 confocal microscope, then 10 UM of c1, c2, or DOX was added and images were taken every 10 min (averaged every 30 min) for 18 h. Approximately 20 cells present in each field of view were averaged for each sample and time point; normalized and averaged data from two replicates each±SEM were included for c1 and DOX. An earlier independent trial yielded very similar results (Fig. S1).

FIG. 5. Cytotoxicity of DOX and prodrugs in H9C2 cardiomyoblasts. Media from cells exposed for 24 or 48 h to 10 or 20 UM of c1, c2, DOX or vehicle (DMSO, final concentration 0.1% in all samples) was centrifuged to remove cells and cell debris, and supernatants were assessed spectrophotometrically by lactose dehydrogenase (LDH) assay to evaluate release into the media as a measure of cytotoxicity (n=6 or more). *, p<0.05; **, p<0.01; ***, p<0.001; ****, p<0.0001.

FIG. 6. Caspase cleavage monitored by an antibody against total caspase-3 demonstrates the H₂S-dependent protection exhibited by c1 against DOX-mediated apoptotic signaling. H9C2 cells in culture were treated for 24 hr with c1, c2, DOX or vehicle (DMSO), then harvested into lysis buffer and immunoblotted for caspase-3. Data analyzed by ImageJ were used to assess the percent of the two bands present as the lower band. A. At 24 hr, only DOX treatment causes caspase-3 cleavage (n=3). B. When 100 mM hydroxocobalamin is added 5 min prior to 10 UM drug treatments, c1-treated cells exhibit much more cleavage of caspase-3 than in its absence, whereas cleavage due to DOX treatment is unchanged (n=7); ***, p<0.001; ****, p<0.0001.

FIG. 7. c1-treated cardiomyocytes do not shut down Nrf2 activation as DOX does. Immunoblots for the transcriptional regulator Nrf2 (left) and one of its downstream targets, HO-1 (right), demonstrate stabilization of Nrf2 with concomitant expression of HO-1 in DMSO-treated samples; DOX treatment completely suppressed both, while c1 treatment was only moderately suppressive (n=4 and n=3 for Nrf2 and HO-1, respectively). The bar graphs below represent the mean±SEM. Results were statistically different between c1 and DOX in both cases (p=0.002 and p=0.019), and more marginally so between c1 and DMSO (p=0.011 and p.=0.025), for Nrf2 and HO-1, respectively.

FIG. 8. Cytotoxicity of DOX and prodrugs in 4T1 mouse breast cancer cells. As in FIG. 5, supernatants of media from cells exposed for 24 or 48 h to 10 or 20 UM of c1, c2, DOX or vehicle) were assessed spectrophotometrically by lactate dehydrogenase (LDH) assay to evaluate release into the media as a measure of cytotoxicity (n=8 or more). *, p<0.05; **, p<0.01; ***, p<0.001; ****, p<0.0001.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment.

Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even±0.1% of the specified value as well as the specified value. For example, “about X” where X is the measurable value, is meant to include X as well as variations of ±10%, ±5%, ±1%, ±0.5%, or even±0.1% of X. A range provided herein for a measurable value may include any other range and/or individual value therein.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein in the accompanying chemical structures, “H” refers to a hydrogen atom. “C” refers to a carbon atom. “N” refers to a nitrogen atom. “S” refers to a sulfur atom. “O” refers to an oxygen atom. “B” refers to a boron atom.

As understood in the art, the term “optionally substituted” indicates that the specified group is either unsubstituted or substituted by one or more suitable substituents. A “substituent” that is “substituted” is a group which takes the place of one or more hydrogen atoms on the parent organic molecule.

“Alkyl,” as used herein, refers to a saturated straight or branched chain, or cyclic hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like.

“Heteroalkyl,” as used herein, means an alkyl, as defined herein, where at least one carbon atom is replaced with a heteroatom selected from the group consisting of O, N, Si and S, wherein the nitrogen and sulfur atoms may optionally be oxidized, and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, naphthyl, azulenyl, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzimidazole, benzothiazole, quinoline, isoquinoline, quinazoline, quinoxaline, phenyl-pyrrole, phenyl-thiophene, etc.

Boronate or diaminoboryl-containing groups such as boronate esters, as used herein, are represented by the following general formula:

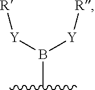

wherein each Y is independently O or NRₐ; and R′, R″, and Rₐ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, —OH, alkoxy, carbonyl and/or carboxy groups. For additional groups that may be substituted on the boronate or diaminoboryl-containing groups, see US 2020/0172561 to Burn et al. In particular embodiments, the groups R′ and R″ together with the Y atoms to which they are attached form a stable cyclic boronate or diaminoboryl group. In some embodiments, R′ and R″ together form a ring that includes 2-5 carbon atoms that is optionally substituted such as with a carbon containing group (e.g., lower alkyl).

Non-limiting examples of suitable boronate or diaminoboryl groups include, but are not limited to, A, B, C, D and E:

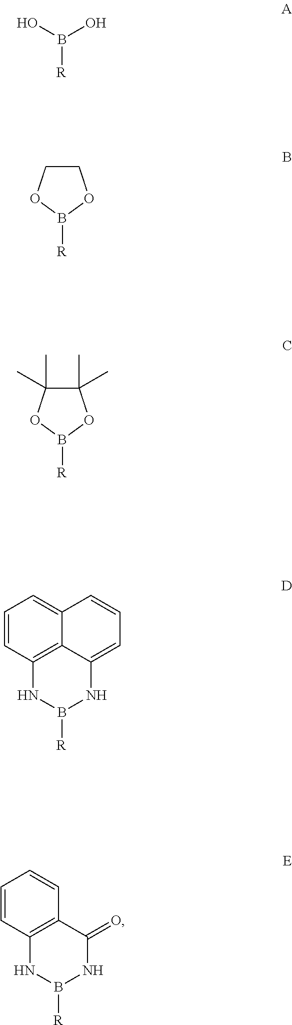

wherein R denotes attachment to the remainder of the molecule. The dimethylboronate ester (moiety C) may be easily hydrolyzed and hence, in some embodiments, the R′ and R″ together with the Y atoms to which they are attached may form moiety C, also referred to as a pinacol ester moiety.

The terms “halo” and “halogen,” as used herein, included fluoro (—F), chloro (—Cl), bromo (—Br), and iodo (—I).

The term “hydroxy,” as used herein, refers to the group —OH.

The term “carboxy,” as used herein, refers to the group —C(═O) OH.

The term “carbonyl,” as used herein, refers to the group —C(═O)—.

The term “alkoxy,” as used herein, refers to an alkyl or loweralkyl group, as defined herein, appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Pharmaceutically acceptable” as used herein means that the compound, carrier, or composition is suitable for administration to a subject to achieve a treatment described herein, without unduly deleterious side effects in light of the severity of the disease and necessity of the treatment.

A “pharmaceutically acceptable salt,” as used herein, is a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with a disease or condition (e.g., cancer) is achieved and/or there is a delay in the progression of the symptom, disease or condition.

A “treatment effective” amount as used herein is an amount that is sufficient to treat (as defined herein) a subject. Those skilled in the art will appreciate that the therapeutic effects need not be complete or curative, as long as some benefit is provided to the subject.

The present invention finds use in both medical and veterinary applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like. Human subjects of all genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects.

Reactive oxygen species (ROS), as used herein, refer to partially reduced or excited forms of oxygen, such as, for example, peroxides (e.g., hydrogen peroxide), superoxide, hydroxyl radical, singlet oxygen, and alpha-oxygen.

The compounds described herein may be formulated as a composition for administration in a pharmaceutically acceptable carrier in accordance with known techniques. See, e.g., Remington, The Science and Practice of Pharmacy (9th Ed. 1995). The carrier may be a solid or a liquid, or both.

Formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration. Parenteral administration includes administration directly into a tumor or a tumor resection cavity.

Formulations of the present invention suitable for parenteral administration comprise sterile aqueous and non-aqueous injection solutions of the compound, which preparations are preferably isotonic with the blood of the intended recipient. These preparations may contain anti-oxidants, buffers, bacteriostats and solutes that render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. When the compound is substantially water-insoluble, a sufficient amount of emulsifying agent that is physiologically acceptable may be employed in sufficient quantity to emulsify it in an aqueous carrier. One such useful emulsifying agent is phosphatidyl choline.

Further, the present invention provides liposomal formulations of the compounds disclosed herein and compositions thereof. The technology for forming liposomal suspensions is well known in the art. When the compound or composition is an aqueous-soluble composition, using conventional liposome technology, the same may be incorporated into lipid vesicles. In such an instance, due to the water solubility of the compound or composition, the compound or composition will be substantially entrained within the hydrophilic center or core of the liposomes. The lipid layer employed may be of any conventional composition and may either contain cholesterol or may be cholesterol-free. When the compound or composition is water-insoluble, again employing conventional liposome formation technology, the compound or composition may be substantially entrained within the hydrophobic lipid bilayer that forms the structure of the liposome. In either instance, the liposomes that are produced may be reduced in size, as through the use of standard sonication and homogenization techniques.

In some embodiments, the compound or composition is provided in a pegylated (polyethylene glycol coated) liposome formulation.

Liposomal formulations containing the compounds disclosed herein or compositions thereof may be lyophilized to produce a lyophilizate, which may be reconstituted with a pharmaceutically acceptable carrier, such as water or saline, to regenerate a liposomal suspension.

Other pharmaceutical compositions may be prepared from water-insoluble compounds disclosed herein, or compositions thereof, such as aqueous base emulsions. In such an instance, the composition will contain a sufficient amount of pharmaceutically acceptable emulsifying agent to emulsify the desired amount of the compound. Particularly useful emulsifying agents include phosphatidyl cholines, and lecithin.

Nanoparticles include particles that are about 0.5 to about 1,000 nanometers in size and may include natural and/or synthetic moieties. See, e.g., U.S. Pat. No. 8,535,726 to Dai et al.; U.S. Pat. No. 8,252,338 to Forte et al.; U.S. Pat. No. 8,246,968 to Zale et al.; U.S. 2013/0122056 to Zhang et al. In some embodiments, the nanoparticle comprises a polymeric matrix, which may comprise two or more polymers. Polymers of the matrix may include, e.g., polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, or combinations thereof. In some embodiments, the polymeric matrix comprises one or more polyesters, polyanhydrides, polyethers, polyurethanes, polymethacrylates, polyacrylates or polycyanoacrylates. In some embodiments, at least one polymer is a polyalkylene glycol. In some embodiments, the polyalkylene glycol is polyethylene glycol. In some embodiments, at least one polymer is a polyester. In some embodiments, the polyester is selected from the group consisting of PLGA, PLA, PGA, and polycaprolactones. In some embodiments, the polyester is PLGA or PLA. In some embodiments, the polymeric matrix comprises a copolymer of two or more polymers, such as a copolymer of a polyalkylene glycol and a polyester. In some embodiments, the copolymer is a copolymer of PLGA or PLA and PEG. In some embodiments, the polymeric matrix comprises PLGA or PLA and a copolymer of PLGA or PLA and PEG.

The compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well-known in the art.

The therapeutically effective dosage will vary somewhat from compound to compound, and patient to patient, and will depend upon factors such as the age and condition of the patient and the route of delivery. Such dosages can be determined in accordance with routine pharmacological procedures known to those skilled in the art.

As a general proposition, the initial pharmaceutically effective amount of the compound administered parenterally will be in the range of about 0.1 to 50 mg/kg of patient body weight per day, with the typical initial range used being 0.3 to 20 mg/kg/day, more preferably 0.3 to 15 mg/kg/day. The desired dosage can be delivered by a single bolus administration, by multiple bolus administrations, or by continuous infusion administration of compound, depending on the pattern of pharmacokinetic decay that the practitioner wishes to achieve.

Subjects treated by the methods of the present invention can also be administered one or more additional therapeutic agents. See U.S. Pat. No. 5,677,178. Chemotherapeutic agents may be administered by methods well known to the skilled practitioner, including systemically, direct injection into the cancer, or by localization at the site of the cancer by associating the desired chemotherapeutic agent with an appropriate slow-release material or intra-arterial perfusing of the tumor. The preferred dose may be chosen by the practitioner based on the nature of the cancer to be treated, and other factors routinely considered in administering. See, e.g., U.S. Pat. No. 7,078,030.

The present invention is further described in the following non-limiting examples.

### EXAMPLES

Herein, we describe a H₂O₂-responsive doxorubicin (DOX) hybrid codrug (mutual prodrug) that has been rationally designed to concurrently liberate hydrogen sulfide (H₂S), a purported cardioprotectant with anticancer activity, in an effort to maintain the antitumor effects of DOX while simultaneously reducing its cardiotoxic side effects.

Experiments with cardiomyoblast cells in culture demonstrated a rapid accumulation of prodrug into the cells, but diminished apoptotic effects compared with DOX, dependent upon its release of H₂S. Cells treated with the prodrug exhibited significantly higher Nrf2 activation relative to DOX-treated cells.

Preliminary indications, using a mouse triple-negative breast cancer cell line sensitive to DOX treatment, are that the prodrug maintains considerable toxicity against the tumor-inducing cell line, suggesting significant promise for this prodrug as a cardioprotective chemotherapeutic to replace DOX.

Shown below is the DOX hybrid prodrug (c1, Scheme 1, panel A) that has been rationally designed to concurrently liberate both hydrogen sulfide (by way of COS hydrolysis) and doxorubicin in response to elevated levels of ROS (Scheme 1, panel B). This design imparts both tumor-selective activation and H₂S delivery as a synergistic strategy to combat DOX-induced cardiotoxicity. This unique combination was shown to afford impressive cardioprotective effects in H₉C2 (rat cardiomyoblast) cells while maintaining the antitumor activity of DOX in 4T1 (mouse triple-negative breast cancer) cells.

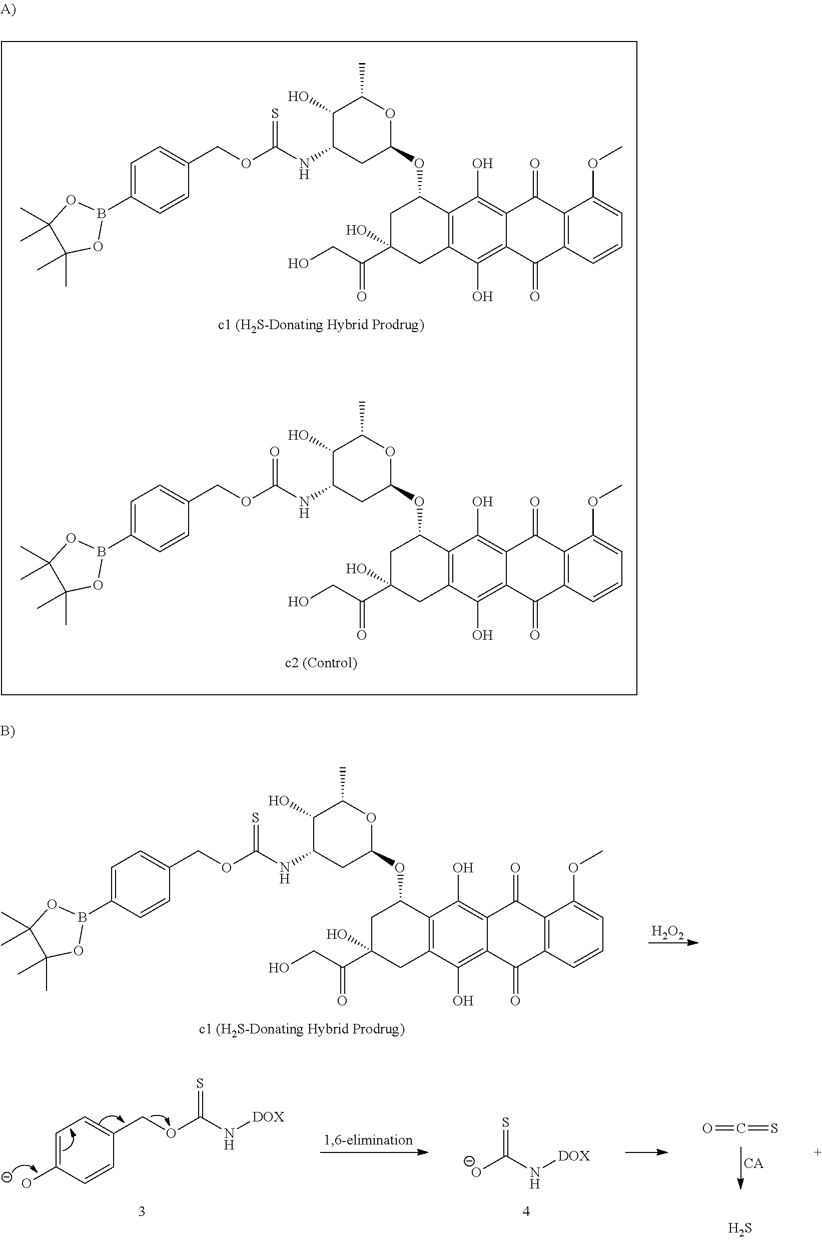

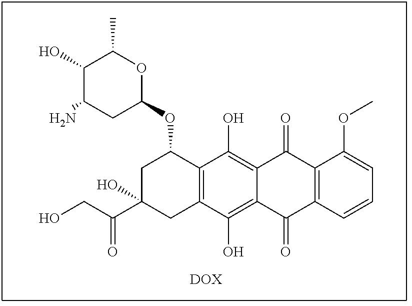

Scheme 1. Prodrug structures and H₂O₂-dependent release pathway, (A) Prodrugs assessed in this study. (B) Proposed mechanism for the simultaneous release of H₂S and DOX from c1 in response to H₂O₂. CA=carbonic anhydrase.

### Chemical Synthesis and Characterization

Synthesis. We successfully accessed c1 in a highly efficient, two-step synthesis. Initially, we coupled 4-(hydroxymethyl)phenylboronic acid pinacol ester with di(2-pyridyl) thionocarbonate. The resulting activated thionocarbonate was treated with DOX and triethylamine to furnish c1 in a 48% yield. As a control, we also generated c2 via an analogous route. Like c1, c2 was predicted to function as a ROS-activated DOX prodrug. However, unlike c1, c2 could not to liberate H₂S alongside DOX.

Time-dependent DOX release from c1. Boronate oxidation is a bioorthogonal reaction that has been employed since the early 2000s to investigate the chemical biology of H₂O₂ under physiological conditions.⁴³ As depicted in Scheme 1B, we surmised that c1, in the presence of H₂O₂, would undergo rapid boronate ester oxidation to yield a phenol, which would then self-immolate via a well-established 1,6-elimination⁴⁴ to release H₂S (Via COS hydrolysis) and free DOX.

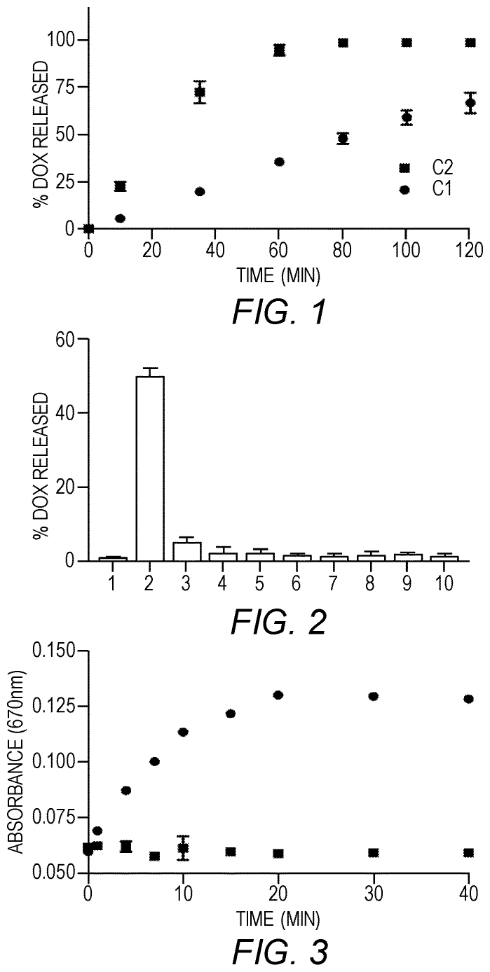

To confirm this reactivity, we incubated c1 (10 μM) with H₂O₂ (10 μM) at room temperature in ammonium bicarbonate buffer (0.1 M, pH 7.4). At various time points, an aliquot was removed and analyzed by LCMS. Using a calibration curve, we determined the percentage of released DOX over the course of two hours. As depicted in FIG. 1, c1 displayed good reactivity towards peroxides, releasing free DOX in yield greater than 70% within the allotted time frame. For comparison, we also assessed the reactivity of c2 towards the same concentration of peroxides. Like c1, c2 also functioned as an H₂O₂-activated DOX prodrug, but with faster kinetics, as quantitative release of free DOX was realized within 60 min. This result, however, is not unexpected given that the carbamate functional group of c2 is likely to increase rates of both the 1,6-elimination and the breakdown of the ensuing carbamic acid to liberate CO₂ and free DOX.⁴⁵ However, the slow and sustained release of DOX from c1, confirmed its potential to serve as an H₂O₂-activated, bifunctional prodrug.

Selectivity of c1. To assess selectivity, c1 was incubated with various biological analytes at room temperature and in ammonium bicarbonate buffer (0.1 M, pH 7.4). After a reaction time of 80 min, an aliquot was removed and analyzed by LCMS. As highlighted in FIG. 2, c1 was shown to be relatively stable in buffer alone and while in the presence of reductants (cysteine, homocysteine, and glutathione) and other oxidants (glutathione disulfide, sodium nitrite, sodium hypochlorite, superoxide, and peroxynitrite). However, in the presence of H₂O₂, significant levels of free DOX were observed by LCMS. The superb stability of c1 in the presence of strong nucleophiles confirms our proposed mechanism of DOX release.

H₂S release from c1. We confirmed the time-dependent liberation of H₂S using a methylene blue assay⁴⁶ and measuring the resulting absorbance at 670 nm after c1 exposure to H₂O₂ (FIG. 3, circle). Using a calibration curve generated with Na₂S, we determined that c1 (40 μM) was able to release more than 16 μM H₂S while in the presence of both H₂O₂ (40 M) and carbonic anhydrase (CA). Conversely, negligible amounts of H₂S were observed in the absence of peroxides (FIG. 3, square). As a control, the same assay was also run in the absence of CA (data not shown). Under these conditions, c1 exposure to H₂O₂ yielded small amounts of H₂S (<5 M). This is presumably due to isocyanate formation and the direct release of H₂S from the breakdown of 4. Therefore, these results not only confirm the efficient release of H₂S from c1 under conditions of oxidative stress, but they also verify that the predominant route of H₂S production (~70%) is via COS hydrolysis facilitated by CA.

### Biological Analyses

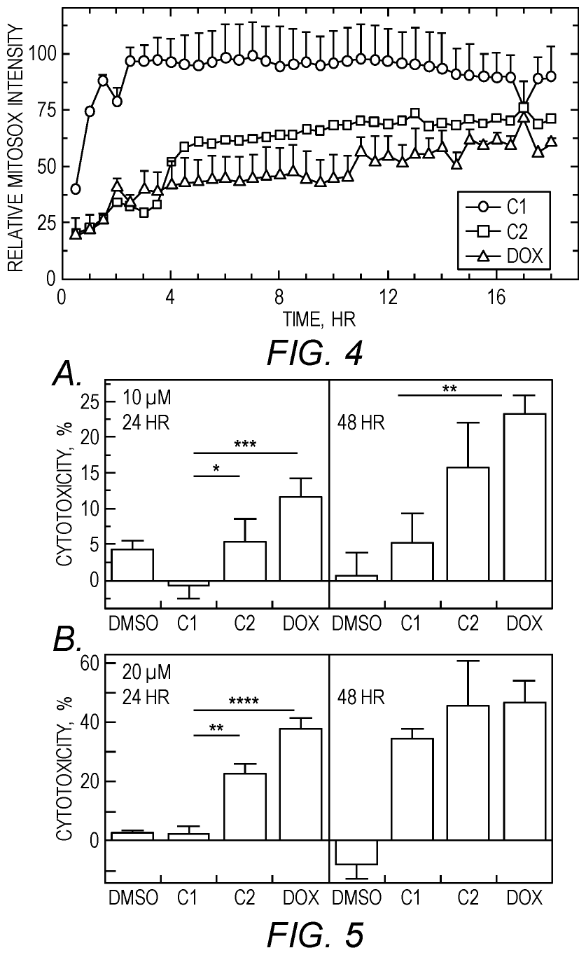

Uptake of DOX and prodrugs into H₉C2 cardiomyoblasts. Before comparing the biological effects of treatments by DOX and prodrugs, it was important to know their time dependence of accumulation into the H9C2 cardiomyoblasts under investigation. For this, confocal microscopy of live cells in culture was used to track in real time the accumulation of the fluorescent molecules into the cells, allowing direct comparisons between c1, c2 and Dox. In both trials shown in FIG. 4, c1 accumulated rapidly in cells (giving maximal signal by ~2 h) compared with DOX. On the other hand, c2 was as slow as or slower than Dox in being taken up by these cells. Thus, any biological effects observed using c1 rather than DOX will not be due to limited cellular uptake.

H₂S-dependent suppression of DOX-triggered apoptosis by use of c1 in lieu of the parent drug, DOX. Cytotoxicity of c1, c2 and DOX was evaluated by measuring the release of lactate dehydrogenase (LDH) into the culture medium over time. Even though c1 was the fastest to accumulate in cells based on our confocal microscopy data (FIG. 4), DOX was more toxic than c1 at both doses, 10 and 20 μM. However, the difference was not significant at the longer exposure time (48 h) (FIG. 5). c2, which releases DOX but not H₂S and is slow to enter cells, was of intermediate toxicity but more similar to DOX than c1.

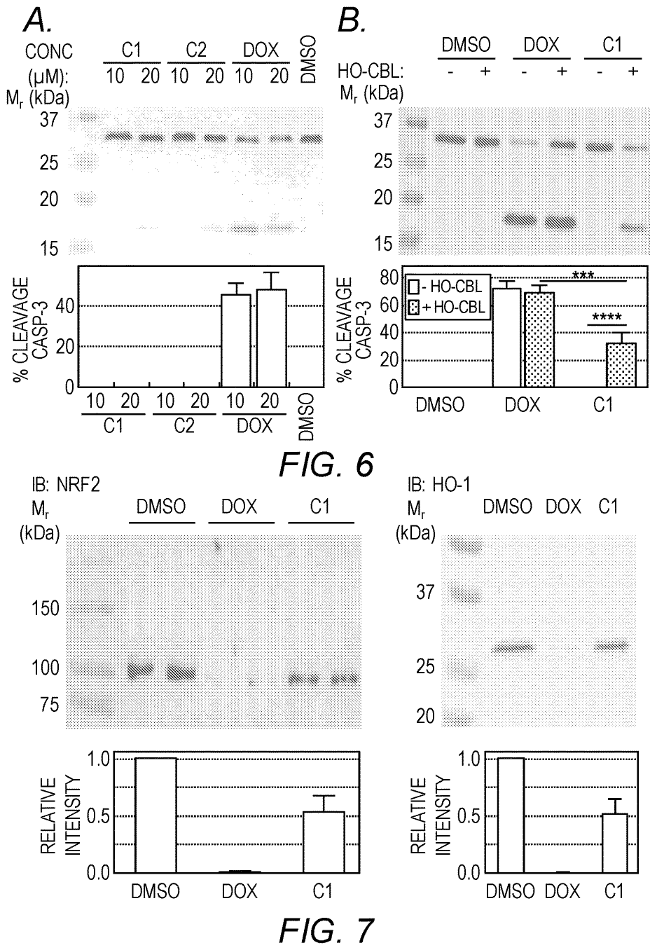

We next evaluated apoptotic signaling through caspase-3 cleavage at 24 h to compare the experimental compounds. Unlike DOX, neither c1 nor c2 at either 10 or 20 UM caused notable caspase-3 cleavage within 24 h (FIG. 6, panel A). Use of a scavenger of H₂S, hydroxocobalamin (HO-Cbl),⁴⁷ provided evidence that the protective effect of this DOX-releasing prodrug, c1, is substantially dependent on its ability to release H₂S (FIG. 6, panel B).

Unlike DOX, c1 treatment preserves nuclear factor erythroid 2-related factor 2 (Nrf2) activation and antioxidant enzyme expression while eliciting lower levels of mitochondrial ROS. Nrf2 is a known transcriptional regulator activated when Keap-1 senses oxidants and electrophiles, leading to induced expression of antioxidant enzymes; activation of Nrf2 is a key factor in cardioprotection from anthracycline toxicity in vivo.^{48,49} Our data using DOX-treated cardiomyoblasts, consistent with studies by others,^{50,51} confirmed the suppression of both Nrf2 activation and expression of a downstream target, heme-oxygenase 1 (HO-1), relative to the vehicle control; c1, on the other hand, largely preserved Nrf2 activation and HO-1 expression, indicating that activation of the Nrf2 transcriptome is a likely mechanism involved in the protection of cardiomyoblasts against toxicity observed by treatment with c1 rather than DOX (FIG. 7).

4T1 Mouse Breast Cancer Cells in Culture Exhibit Substantial Cytotoxicity when Treated with c1.

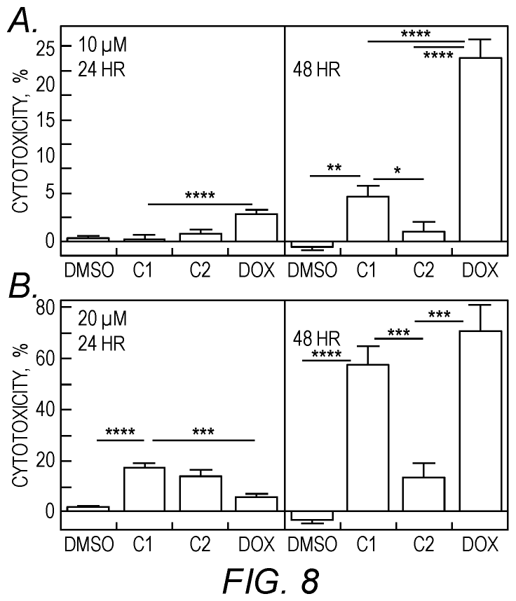

DOX is a known cytotoxic agent used against 4T1 triple-negative breast cancer cells grown in culture or injected orthotopically into mouse mammary fat pads to initiate the development of tumors.^{52,53} As an initial test to see if c1 could retain the cytotoxic properties of DOX toward tumor-forming 4T1 cells, we conducted LDH assays to assess cytotoxicity of DOX, c1 and c2 treatments. Other than the lowest dose and shortest time, depending on the concentration and time of exposure, c1 provokes comparable, sometimes less and sometimes more, toxicity relative to DOX in 4T1 cells, perhaps owing to the anticancer activity of H₂S in combination with DOX (FIG. 8).

### Conclusions

In this study, we disclosed a hybrid prodrug (c1) that was shown to selectively release both DOX and H₂S upon exposure to H₂O₂. We found that c1 accumulates relatively rapidly in cardiomyoblasts but has diminished apoptotic effects compared with DOX, dependent upon its release of H₂S. These c1-treated cells exhibit higher Nrf2 and HO1 levels than DOX-treated cells. Preliminary indications, using a mouse triple-negative breast cancer cell line sensitive to DOX treatment, are that c1 maintains toxicity against this cell line, although with somewhat altered time dependence that may stem, in part, from its facile accumulation in cells. Although not a part of the present study, it has been shown that H₂S production concomitant with DOX release impeded efflux of the drug from a DOX-resistant sarcoma cell line, suggesting selectively toxic effects of H₂S co-production on some treatment-resistant cancers.²¹

Taken together, our results indicate that DOX prodrugs that impart tumor-selective activation, along with H₂S delivery, provide a highly promising and synergistic strategy for combating DOX-induced cardiotoxicity.

This new design strategy will be further evaluated in an in vivo mouse model.

### Experimental Section

### I. General Chemistry

Commercial reagents were used without further purification unless stated otherwise. Dichloromethane and tetrahydrofuran (THF) were dried over a column of alumina. Flash chromatography was performed with columns of 40-63 Å silica from Silicycle (Québec City, Canada). Thin-layer chromatography (TLC) was performed on plates of EMD 250 μm silica 60-F₂₅₄. The term “concentrated under reduced pressure” refers to removing solvents and other volatile materials using a rotary evaporator while maintaining the water-bath temperature below 40° C. Residual solvent was removed from samples at high vacuum (<0.1 torr) using an Edwards RV5 pump. Analytically pure samples of final products were accessed using an Agilent (Santa Clara, CA) preparative HPLC, equipped with a C18 reverse-phase preparative column, diode array detector, and fraction collector. Liquid Chromatography-Mass Spectrometry (LC-MS) analyses were performed using a Bruker AmaZon SL with a Shimadzu SPD-M20A UV detector and a Shimadzu LC-20AB pump, equipped with an analytical C18 column (Agilent Technologies, SB—C18 Analytical HPLC Col. 4.6×150). All NMR spectra were acquired at ambient temperature with a Bruker Ascend™ 400 MHz spectrometer and referenced to TMS or residual protic solvent. High-resolution mass spectra were acquired using a Thermo Orbitrap LTQ XL (ESI). Carbonyl sulfide liberation was detected using an Agilent 7890 GC/5975 MS with autosampler. Absorbance measurements for the methylene blue assay were taken with a Cary 100 UV-Vis spectrophotometer (Agilent). All data fitting was done with Prism 7 (GraphPad Software, La Jolla, Ca). All cell imaging was acquired using a Carl Zeiss Laser Scanning Confocal Microscope 880 with Airyscan (Oberkochen, Germany).

### II. Chemical Synthesis

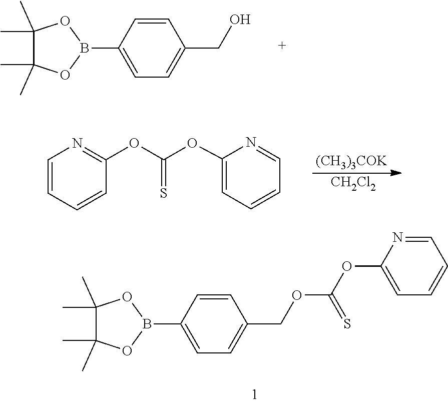

Potassium tert-butoxide (1.5 mL, 1M in THF) was added dropwise to a solution of 4-(hydroxymethyl)phenylboronic acid pinacol ester (234 mg, 1.0 mmol) in dry CH₂Cl₂ (5 mL). The reaction mixture was then taken up with a syringe and added dropwise over a period of 5 min to a solution of di(2-pyridyl) thionocarbonate (464 mg, 2.0 mmol) dissolved in dry CH₂Cl₂ (5 mL). After reacting for 15 min under N₂ (g), the reaction was confirmed to be complete by TLC, and the reaction mixture was diluted with CH₂Cl₂, washed with 1M HCl (aq), dried over anhydrous MgSO₄, and concentrated under reduced pressure. Flash chromatography (15% v/v EtOAc in Hexanes) was used to isolate 1 (185 mg) in a 50% yield.

¹H NMR (400 MHz, Chloroform-d) δ 7.83 (d, J=8.1 Hz, 2H), 7.60 (ddd, J=7.2, 2.1, 1.3 Hz, 1H), 7.48 (d, J=8.1 Hz, 2H), 7.26 (ddd, J=9.5, 6.5, 2.1 Hz, 1H), 6.49 (dt, J=9.5, 1.3 Hz, 1H), 6.12 (ddd, J=7.2, 6.5, 1.3 Hz, 1H), 5.66 (s, 2H), 1.33 (s, 12H); ¹³C NMR (101 MHz, Chloroform-d) δ 192.6, 160.1, 140.1, 136.5, 135.4, 135.2, 127.8, 122.8, 105.7, 84.0, 76.8, 25.0; ESI-MS calculated for [C₁₉H₂₃BNO₄S]⁺ (M+H)⁺ requires m/z=372.14, found 372.19.

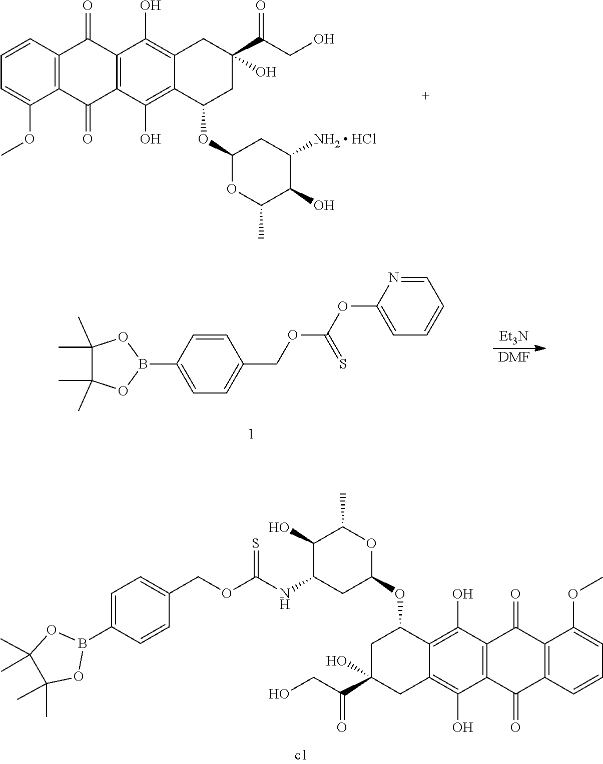

To a stirred solution of doxorubicin hydrochloride (58 mg, 0.1 mmol) in dry DMF (1 mL) was slowly added a mixture of 1 (37 mg, 0.1 mmol) and Et₃N (200 μL, 1.4 mmol) in dry DMF (2 mL). The mixture was then left to react at room temperature, in the dark, and under an N₂ (g) atmosphere. After stirring for 2 h, the reaction mixture was concentrated under reduced pressure and purified via flash chromatography (10% v/v MeOH in DCM) to isolate c1 (78 mg, 95%). An analytically pure sample of c1 (42 mg, 51%) was then obtained via HPLC (Agilent) using a C18 preparatory column and eluting at 20 mL/min with water (0-1 min), followed by a linear gradient (0-100% v/v) of acetonitrile/water (1-9 min), and finishing with an acetonitrile wash (9-12 min).

¹H NMR (400 MHz, Chloroform-d) δ 14.01 (s, 1H), 13.26 (s, 1H), 8.05 (dd, J=7.7, 1.1 Hz, 1H), 7.81-7.77 (m, 3H), 7.41-7.38 (m, 1H), 7.33 (d, J=8.1 Hz, 2H), 6.64 (d, J=8.6 Hz, 1H), 5.55-5.52 (m, 1H), 5.42 (d, J=2.3 Hz, 1H), 5.37-5.34 (m, 1H), 4.80 (d, J=4.7 Hz, 2H), 4.62 (s, 1H), 4.56-4.49 (m, 1H), 4.21-4.16 (m, 1H), 4.09 (s, 3H), 3.79-3.77 (m, 1H), 3.49 (s, 2H), 3.35-3.27 (m, 1H), 3.00 (t, J=4.7 Hz, 1H), 2.38-2.31 (m, 1H), 2.22-2.17 (m, 1H), 2.09-2.04 (m, 1H), 1.92-1.86 (m, 1H), 1.78 (td, J=13.1, 4.1 Hz, 1H), 1.34 (s, 3H), 1.33 (s, 12H); ¹³C NMR (101 MHz, Chloroform-d) δ 214.1, 189.3, 187.3, 186.9, 161.2, 156.3, 155.9, 138.8, 135.9, 135.7, 135.1, 133.9, 133.7, 127.6, 126.9, 121.1, 120.0, 118.6, 111.8, 111.7, 100.3, 84.0, 71.9, 69.2, 69.0, 67.4, 65.8, 56.9, 53.6, 51.2, 35.9, 34.3, 29.1, 25.0, 17.0. HRMS calculated for [C₄₁H₄₆O₁₄NBNaS]⁺ (M+Na⁺) requires m/z=842.2630, found 842.2632. Purity of c1 (retention time 13.7 min) was determined by LC-MS (Shimadzu) using a C18 analytical column and eluting at 0.4 mL/min with a linear gradient (20-90% v/v) MeOH/H₂O (0-2 min), followed by 90% v/v MeOH/H₂O (2-20 min) and found to be >95% pure.

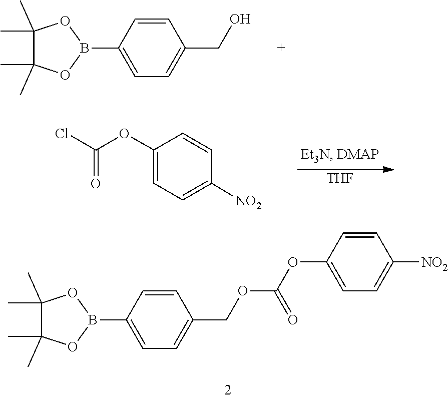 [SMILES: CC1(C)OB(c2ccc(CO)cc2)OC1(C)C ; O=C(Cl)Oc1ccc([N+](=O)[O-])cc1 ; CC1(C)OB(c2ccc(COC(=O)Oc3ccc([N+](=O)[O-])cc3)cc2)OC1(C)C ; "THF"]

4-Nitrophenyl chloroformate (280 mg, 1.4 mmol) was dissolved in dry THF (5 mL) under an N₂ (g) atmosphere. Next, a mixture of 4-(hydroxymethyl)phenylboronic acid pinacol ester (234 mg, 1.0 mmol), DMAP (48 mg, 0.4 mmol), and Et₃N (560 μL, 4.0 mmol) in dry THF (5 mL) was added dropwise. After stirring at room temperature for 4 h, the reaction mixture was concentrated under reduced pressure, diluted with EtOAc, washed with 1M HCl (aq), dried over anhydrous MgSO₄, and rotary evaporated to dryness. Flash chromatography (15% v/v EtOAc in Hex) was used to isolate 2 (288 mg, 72%).

¹H NMR (400 MHz, Chloroform-d) δ 8.25 (d, J=9.2 Hz, 2H), 7.85 (d, J=8.1 Hz, 2H), 7.43 (d, J=8.1 Hz, 2H), 7.37 (d, J=9.2 Hz, 2H), 5.30 (s, 2H), 1.35 (s, 12H); ¹³C NMR (101 MHz, Chloroform-d) δ 155.58, 152.49, 145.46, 137.17, 135.26, 127.69, 125.35, 121.85, 84.06, 70.85, 24.94. MS (ESI): calcd for C₂₀H₂₂BNNaO₇⁺ (M+H)⁺ 422.1382, found 422.21.

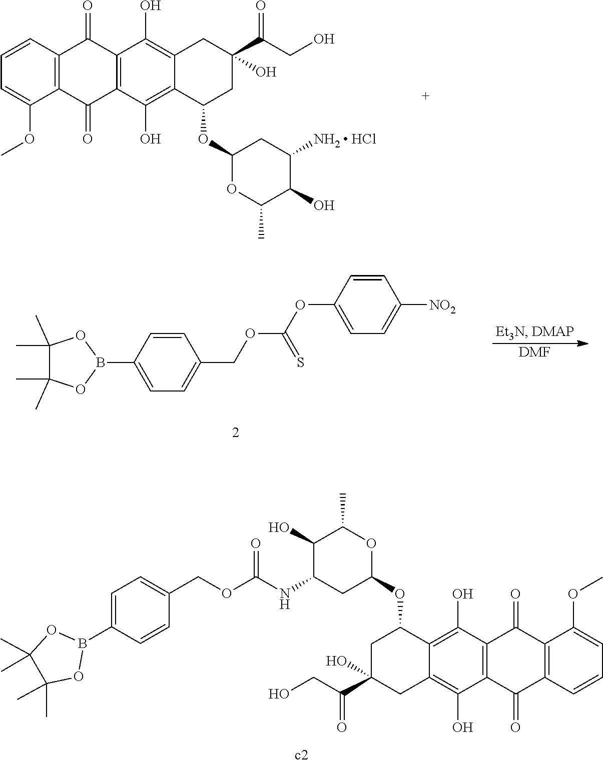

To a stirred solution of doxorubicin hydrochloride (58 mg, 0.1 mmol) in 1 mL of dry DMF was slowly added a mixture of 2 (40 mg, 0.1 mmol), DMAP (5 mg, 0.04 mmol), and Et₃N (20 μL, 0.14 mmol) in DMF (2 mL). The mixture was then left to react at room temperature, in the dark, and under an N₂ (g) atmosphere. After reacting for 2 h, the solvent was then removed via rotary evaporation, and flash chromatography (10% v/v MeOH in DCM) was used to isolate c2 (75 mg, 94%). An analytically pure sample of c2 (33 mg, 41%) was then obtained via HPLC (Agilent) using a C18 preparatory column and eluting at 20 mL/min with water (0-1 min), followed by a linear gradient (0-100% v/v) of acetonitrile/water (1-9 min), and finishing with an acetonitrile wash (9-12 min).

¹H NMR (400 MHz, Chloroform-d) δ 13.97 (s, 1H), 13.23 (s, 1H), 8.03 (d, J=7.8 Hz, 1H), 7.84-7.75 (m, 3H), 7.39 (d, J=8.6, 1.1 Hz, 1H), 7.29 (d, J=7.6 Hz, 2H), 5.50 (d, J=3.9 Hz, 1H), 5.18-5.09 (m, 1H), 5.04 (s, 2H), 4.80-4.70 (m, 2H), 4.54 (s, 1H), 4.15-4.11 (m, 1H), 4.08 (s, 3H), 3.95-3.77 (m, 1H), 3.71-3.62 (m, 1H), 3.48 (s, 1H), 3.27 (dd, J=17.0, 1.9 Hz, 1H), 3.09-2.92 (m, 2H), 2.33 (dt, J=14.7, 2.1 Hz, 1H), 2.23-2.10 (m, 1H), 2.03-1.83 (m, 2H), 1.77 (td, J=13.2, 4.1 Hz, 1H), 1.63 (s, 3H), 1.32 (s, 12H); ¹³C NMR (101 MHz, Chloroform-d) δ 214.0, 187.3, 186.9, 161.2, 156.3, 155.8, 155.6, 139.5, 135.9, 135.7, 135.1, 133.7, 127.3, 121.0, 120.0, 118.6, 111.8, 111.6, 100.8, 84.0, 69.7, 67.4, 66.8, 65.7, 56.8, 53.6, 47.1, 35.8, 34.2, 30.3, 29.8, 25.0, 22.8, 17.0, 14.3; HRMS calculated for [C₄₁H₄₆O₁₅NBNa]⁺ (M+Na⁺) requires m/z=826.2858, found 826.2868. Purity of c2 (retention time 11.9 min) was determined by LC-MS (Shimadzu) using a C18 analytical column and eluting at 0.4 mL/min with a linear gradient (20-90% v/v) MeOH/H₂O (0-2 min), followed by 90% v/v MeOH/H₂O (2-20 min) and found to be >95% pure.

### III. H₂S Calibration Curve Using the Methylene Blue (MB) Assay

With carbonic anhydrase: Six separate vials were each filled with 150 μL of freshly degassed PBS (pH 7.4) containing Zn(OAc)₂ (2 mM), carbonic anhydrase (0.1 mg/mL), and DOX (80 μM). Next, 150 μL of Na₂S stock solution in freshly degassed PBS (pH 7.4), and at differing concentrations (6.25, 12.5, 25.0, 50.0, 100 and 200.0 μM), was added to each vial to give a final volume of 300 μL and a final concentration of 3.125, 6.25, 12.5, 25.0, 50.0, and 100.0 UM Na₂S. Next, 600 μL of MB cocktail (300 μL FeCl₃ (30.0 mM in 1.20 M HCl) and 300 μL N,N-dimethyl-p-phenylene diamine (20.0 mM in 7.20 M HCl)) was added to each vial and allowed to react for 30 min in the dark. The MB solution was transferred to a 1.0 mL UV cuvette and the absorbance at 670 nm was recorded.

Without carbonic anhydrase: Six separate vials were each filled with 150 μL of freshly degassed PBS (pH 7.4) containing Zn(OAc)₂ (2 mM) and DOX (80 μM). Next, 150 μL of Na₂S stock solution in freshly degassed PBS buffer (pH 7.4), and at differing concentrations (6.25, 12.5, 25.0, 50.0, 100 and 200.0 M), was added to each vial to give a final volume of 300 μL and a final concentration of 3.125, 6.25, 12.5, 25.0, 50.0, and 100.0 μM Na₂S. Next, 600 μL of MB cocktail (300 μL FeCl₃ (30.0 mM in 1.20 M HCl) and 300 μL N,N-dimethyl-p-phenylene diamine (20.0 mM in 7.20 M HCl)) was added to each vial and allowed to react for 30 min in the dark. The MB solution was transferred to a 1.0 mL UV cuvette and the absorbance at 670 nm was recorded.

### IV. Time-Dependent H₂S Release from c1

A 10 mM stock solution of c1 (or c2) was prepared in DMSO immediately prior to use. Hydrogen peroxide (H₂O₂, 10 mM) was prepared in freshly degassed PBS (pH 7.4). Carbonic anhydrase (CA) was prepared as 10 mg/mL in PBS buffer (pH 7.4), Zn(OAc)₂ (100 mM) was prepared in distilled water), and methylene blue cocktail was prepared as follows: FeCl₃ (20 mM, 1.2 M HCl) and N,N-dimethyl-p-phenylene diamine (20 mM, 7.2 M HCl).

With carbonic anhydrase: To a 20 mL scintillation vial containing 9770 μL of freshly degassed PBS (pH 7.4) was added stock solutions of carbonic anhydrase (50 μL), H₂O₂ (40 μL), Zn(OAc)₂ (100 μL), and c1 (or c2) (40 μL). The resulting mixture was then stirred at 37° C., and at various time points (1 min, 10 min, 20 min, 30 min, 40 min, 50 min, 60 min, 75 min, 90 min, 105 min, 120 min), a 300 μL aliquot was removed and added to the MB solution (300 μL FeCl₃ and 300 μL N,N-dimethyl-p-phenylene diamine). After reacting for 30 min in the dark, absorbance measurements were recorded at 670 nm.

Without carbonic anhydrase: To a 20 mL scintillation containing 9820 μL of freshly degassed PBS (pH 7.4) was added stock solutions of H₂O₂ (40 μL), Zn(OAc)₂ (100 μL), and c1 (or c2) (40 μL). The resulting mixture was then stirred at 37° C., and at various time points (1 min, 10 min, 20 min, 30 min, 40 min, 50 min, 60 min, 75 min, 90 min, 105 min, 120 min), a 300 μL aliquot was removed and added to the MB solution (300 μL FeCl₃ and 300 μL N,N-dimethyl-p-phenylene diamine). After reacting for 30 min in the dark, absorbance measurements were recorded at 670 nm.

### V. Time-Dependent Dox Release from c1

A stock solution of c1 (or c2, 10 mM) in DMSO and H₂O₂ (10 mM) in ammonium bicarbonate buffer (0.1M, pH 7.4) were prepared immediately prior to use. To a 20 mL scintillation vial containing ammonium bicarbonate buffer (9980 μL) was then added H₂O₂ (10 μL) and c1 (10 μL), and the reaction mixture was stirred at 37° C. At various time points (0 min, 10 min, 35 min, 60 min, 80 min, 100 min, 120 min), a 1 mL (inject volume 60 μL) aliquot was removed and analyzed by LC-MS (using a C18 analytical column and eluting at 0.4 mL/min with a linear gradient (20-90% v/v) MeOH/H₂O (0-2 min), followed by 90% v/v MeOH/H₂O (2-19 min)). The signals in the chromatogram were recorded at 500 nm and the peak corresponding to free DOX (7.0 min) was integrated. A calibration was generated and used to determine the concentration of released DOX from c1 (or c2) at each time point.

### VI. Selectivity Studies for DOX Release from c1

Analyte stock solutions of glutathione (10 mM), oxidized glutathione (10 mM), homocysteine (10 mM), L-cysteine (10 mM), sodium nitrite (NaNO₂ 10 mM), superoxide (KO₂, 10 mM), sodium peroxynitrite (NaOONO, 10 mM), and sodium hypochlorite (NaClO, 10 mM), were prepared in ammonium bicarbonate buffer (0.1 M pH 7.4). To a 20 mL scintillation vial was then added 9980 μL of ammonium bicarbonate buffer (pH 7.4) and 10 μL of analyte and c1 stock solutions, and the resulting mixture was stirred at 37° C. After reacting for 80 min, a 1 mL (inject volume 60 μL) aliquot was removed and analyzed by LC-MS (using a C18 analytical column and eluting at 0.4 mL/min with a linear gradient (20-90% v/v) MeOH/H₂O (0-2 min), followed by 90% v/v MeOH/H₂O (2-19 min)). The signals in the chromatogram were recorded at 500 nm and the peak corresponding to free DOX (7.0 min) was integrated.

### VII. Cell Culture

4T1 mouse breast cancer cells were a gift to D.S.-P. from Dr. Patricia Steeg [National Cancer Institute (NCI, National Institutes of Health (NIH) Bethesda, Maryland). H₉C2 rat cardiac myoblast cells were obtained from ATCC (CRL-1446, TIB-71). All cell lines were cultured in DMEM supplemented with 10% fetal bovine serum (FBS) penicillin/streptomycin, and glutamine kept at 37° C. and 5% CO₂.

### VIII. Confocal Microscopy

To evaluate drug uptake, H₉C2 cells were plated at a density of 20,000 per well on 24 well #1.5 polymer chambered coverslips (Ibidi) in media including 10% FBS and allowed to adhere for ~20 h. Prior to imaging analysis, the media was replaced with Fluorobrite DMEM imaging media (Gibco) supplemented with 5% FBS. To 1 mL of media in each well was added either vehicle (DMSO), or a 20 mM stock solution of c1, c2, or DOX to give final concentrations of 10 or 20 UM drug and 0.1% DMSO. Live cell imaging was performed on a Zeiss LSM 880 confocal microscope with standard incubation conditions (37° C., 5% CO₂, humidified), collecting images every 10 or 15 minutes for 18-24 h. Images were captured with a Plan-Apochromat 20×/0.8 air objective. DOX and the prodrugs were detected with excitation by a 458 nm laser at 0.3% and emission from 535-648 nm.

Each position consisted of a 3 slice z-stack that was compressed with a maximum intensity projection for analysis. Cells were identified using the cell count recipe from Aivia 9.8.1. The mean intensity for both channels was calculated, normalized, and then averaged to 30 min time points.

### IX. Cytotoxicity Assays

H₉C2 and 4T1 cells were plated at a density of 10,000 per well in 96-well plates. Twenty-four h after plating, cells were treated with 10 or 20 UM DOX (from 20 mM stock dissolved in DMSO, for 0.1% final DMSO) and were incubated for 24 or 48 h. The extracellular medium was centrifuged at 12,000×g for 5 min to pellet cellular debris, and the supernatants were assessed for release of lactate dehydrogenase (LDH) using a colorimetric activity assay (Invitrogen) as directed and spectral detection (Benchmark or BioRad microplate reader). Values were used to determine % cytotoxicity as described in the kit, using treatment with lysis buffer to release all LDH into the medium (defining 100% release) or cells without treatment (to define 0% release).

### X. Western Blot Analysis

To evaluate levels of caspase cleavage (using an antibody recognizing both cleaved and uncleaved, Cell Signaling, cat #9662), Nrf2 (antibody from Thermo Fisher, cat #PA5-27882), and HO-1 (antibody from Proteintech, cat #10701-1-AP), H9C2 cells were plated at a density of 450,000 per well in a 24-well plate, then treated 24 h later with 10 or 20 UM DOX and incubated another 24 h before harvesting. For some samples, 100 mM hydroxocobalamin (HO-Cbl) was added 5 min before drug treatment. Cells were harvested by removing media (and recovering any non-adherent cells by centrifugation for 5 min at 10,600×g, discarding the supernatant), and combining that with the 120 μL of lysis buffer added to each well. Lysis buffer [50 mM Tris-HCl at pH 8.0, with 100 mM NaCl, 100 UM diethylene triamine pentaacetic acid (DTPA), 20 mM β-glycerophosphate, 0.1% SDS, 0.5% Na Desoxycholate, and 0.5% Triton-X-100] was prepared by freshly adding protease and phosphatase inhibitors before use (1 mM PMSF, 10 μg/mL aprotinin, 1 mM Na₃VO₄, 10 mM NaF and 10 μg/mL leupeptin). Samples were further incubated on ice for 30 min, then centrifuged at high speed (20,800×g for 10 min) to remove cell debris, and supernatants were mixed with 5×SDS protein sample buffer for Western blot analysis following resolution of 40 μg per sample on 10% SDS-polyacrylamide gels. Protein concentrations of supernatants were determined using a BCA assay (Pierce). Antibodies were used at dilutions of 1:1000 (for HO-1 and caspase-3) or 1:3000 (for Nrf2).

### XI. Statistical Analysis

Data are presented as the mean±standard error of the mean. Statistical analyses of data from LDH assays and Western blot intensity data (for caspase-3, Nrf2 and HO-1) were conducted by Student t-test. The criterion for statistical significance was set at p<0.05.

### REFERENCES

(1) Minotti, G.; Menna, P.; Salvatorelli, E.; Cairo, G.; Gianni, L. Anthracyclines: Molecular Advances and Pharmacologic Developments in Antitumor Activity and Cardiotoxicity. Pharmacol. Rev. 2004, 56 (2), 185-229. https://doi.org/10.1124/pr.56.2.6.
(2) Arcamone, F.; Cassinelli, G.; Fantini, G.; Grein, A.; Orezzi, P.; Pol, C.; Spalla, C. Adriamycin, 14-Hydroxydaimomycin, a New Antitumor Antibiotic FromS. Peucetius Var. Caesius. Biotechnol. Bioeng. 1969, 11 (6), 1101-1110. https://doi.org/10.1002/bit.260110607.
(3) Sun, J.; Wei, Q.; Zhou, Y.; Wang, J.; Liu, Q.; Xu, H. A Systematic Analysis of FDA-Approved Anticancer Drugs. BMC Syst. Biol. 2017, 11 (S5), 87. https://doi.org/10.1186/s12918-017-0464-7.
(4) Sritharan, S.; Sivalingam, N. A Comprehensive Review on Time-Tested Anticancer Drug Doxorubicin. Life Sci. 2021, 278, 119527. https://doi.org/10.1016/j.Ifs.2021.119527.
(5) Gewirtz, D. A Critical Evaluation of the Mechanisms of Action Proposed for the Antitumor Effects of the Anthracycline Antibiotics Adriamycin and Daunorubicin. Biochem. Pharmacol. 1999, 57 (7), 727-741. https://doi.org/10.1016/S0006-2952 (98) 00307-4.
(6) Singal, P. K.; Iliskovic, N. Doxorubicin-Induced Cardiomyopathy. N. Engl. J. Med. 1998, 339 (13), 900-905. https://doi.org/10.1056/NEJM199809243391307.
(7) Buzdar, A. U.; Marcus, C.; Blumenschein, G. R.; Smith, T. L. Early and Delayed Clinical Cardiotoxicity of Doxorubicin. Cancer 1985, 55 (12), 2761-2765. https://doi.org/10.1002/1097-0142 (19850615)55:12<2761::AID-CNCR2820551206>3.0.CO;2-P.
(8) Swain, S. M.; Whaley, F. S.; Ewer, M. S. Congestive Heart Failure in Patients Treated with Doxorubicin: A Retrospective Analysis of Three Trials. Cancer 2003, 97 (11), 2869-2879. https://doi.org/10.1002/cncr.11407.
(9) Berthiaume, J. M.; Wallace, K. B. Adriamycin-Induced Oxidative Mitochondrial Cardiotoxicity. Cell Biol. Toxicol. 2007, 23 (1), 15-25. https://doi.org/10.1007/s10565-006-0140-y.
(10) Granados-Principal, S.; Quiles, J. L.; Ramirez-Tortosa, C. L.; Sanchez-Rovira, P.; Ramirez-Tortosa, Mc. New Advances in Molecular Mechanisms and the Prevention of Adriamycin Toxicity by Antioxidant Nutrients. Food Chem. Toxicol. 2010, 48 (6), 1425-1438. https://doi.org/10.1016/j.fct.2010.04.007.
(11) Octavia, Y.; Tocchetti, C. G.; Gabrielson, K. L.; Janssens, S.; Crijns, H. J.; Moens, A. L. Doxorubicin-Induced Cardiomyopathy: From Molecular Mechanisms to Therapeutic Strategies. J. Mol. Cell. Cardiol. 2012, 52 (6), 1213-1225. https://doi.org/10.1016/j.yjmcc.2012.03.006.
(12) Upadhayay, S.; Sharma, N.; Mantha, A. K.; Dhiman, M. ANTI-CANCER DRUG DOXORUBICIN INDUCED CARDIOTOXICITY: UNDERSTANDING THE MECHANISMS INVOLVED IN ROS GENERATION RESULTING IN MITOCHONDRIAL DYSFUNCTION. Rasayan J. Chem. 2020, 13 (02), 1042-1053. https://doi.org/10.31788/RJC.2020.1325603.
(13) Rawat, P. S.; Jaiswal, A.; Khurana, A.; Bhatti, J. S.; Navik, U. Doxorubicin-Induced Cardiotoxicity: An Update on the Molecular Mechanism and Novel Therapeutic Strategies for Effective Management. Biomed. Pharmacother. 2021, 139, 111708. https://doi.org/10.1016/j.biopha.2021.111708.
(14) Doroshow, J. H.; Davies, K. J. Redox Cycling of Anthracyclines by Cardiac Mitochondria. II. Formation of Superoxide Anion, Hydrogen Peroxide, and Hydroxyl Radical. J. Biol. Chem. 1986, 261 (7), 3068-3074. https://doi.org/10.1016/S0021-9258(17)35747-2.
(15) Wallace, K. B.; Sardão, V. A.; Oliveira, P. J. Mitochondrial Determinants of Doxorubicin-Induced Cardiomyopathy. Circ. Res. 2020, 126 (7), 926-941. https://doi.org/10.1161/CIRCRESAHA. 119.314681.
(16) Doroshow, J. H.; Locker, G. Y.; Myers, C. E. Enzymatic Defenses of the Mouse Heart Against Reactive Oxygen Metabolites. J. Clin. Invest. 1980, 65 (1), 128-135. https://doi.org/10.1172/JCI109642.
(17) Quiles, J. Antioxidant Nutrients and Adriamycin Toxicity. Toxicology 2002, 180 (1), 79-95. https://doi.org/10.1016/S0300-483X(02)00383-9.
(18) DeAtley, S. M.; Aksenov, M. Y.; Aksenova, M. V.; Harris, B.; Hadley, R.; Cole Harper, P.; Carney, J. M.; Butterfield, D. A. Antioxidants Protect against Reactive Oxygen Species Associated with Adriamycin-Treated Cardiomyocytes. Cancer Lett. 1999, 136 (1), 41-46. https://doi.org/10.1016/S0304-3835(98)00306-1.
(19) Hideg, K.; Kálai, T. Novel Antioxidants in Anthracycline Cardiotoxicity. Cardiovasc. Toxicol. 2007, 7 (2), 160-164. https://doi.org/10.1007/s12012-007-0019-Z.
(20) Chegaev, K.; Riganti, C.; Rolando, B.; Lazzarato, L.; Gazzano, E.; Guglielmo, S.; Ghigo, D.; Fruttero, R.; Gasco, A. Doxorubicin-Antioxidant Co-Drugs. Bioorg. Med. Chem. Lett. 2013, 23 (19), 5307-5310. https://doi.org/10.1016/j.bmcl.2013.07.070.
(21) Chegaev, K.; Rolando, B.; Cortese, D.; Gazzano, E.; Buondonno, I.;

Lazzarato, L.; Fanelli, M.; Hattinger, C. M.; Serra, M.; Riganti, C.; Fruttero, R.; Ghigo, D.; Gasco, A. H₂S-Donating Doxorubicins May Overcome Cardiotoxicity and Multidrug Resistance. J. Med. Chem. 2016, 59 (10), 4881-4889. https://doi.org/10.1021/acs.jmedchem.6b00184.
(22) Fukuto, J. M.; Carrington, S. J.; Tantillo, D. J.; Harrison, J. G.; Ignarro, L. J.; Freeman, B. A.; Chen, A.; Wink, D. A. Small Molecule Signaling Agents: The Integrated Chemistry and Biochemistry of Nitrogen Oxides, Oxides of Carbon, Dioxygen, Hydrogen Sulfide, and Their Derived Species. Chem. Res. Toxicol. 2012, 25 (4), 769-793. https://doi.org/10.1021/tx2005234.
(23) Cao, X.; Ding, L.; Xie, Z.; Yang, Y.; Whiteman, M.; Moore, P. K.; Bian, J.-S. A Review of Hydrogen Sulfide Synthesis, Metabolism, and Measurement: Is Modulation of Hydrogen Sulfide a Novel Therapeutic for Cancer? Antioxid. Redox Signal. 2019, 31 (1), 1-38. https://doi.org/10.1089/ars.2017.7058.
(24) Kimura, H. Hydrogen Sulfide: Its Production, Release and Functions. Amino Acids 2011, 41 (1), 113-121. https://doi.org/10.1007/s00726-010-0510-x.
(25) Bhatia, M. Hydrogen Sulfide as a Vasodilator. IUBMB Life Int. Union Biochem. Mol. Biol. Life 2005, 57 (9), 603-606. https://doi.org/10.1080/15216540500217875.
(26) Cacanyiova, S.; Berenyiova, A.; Kristek, F. The Role of Hydrogen Sulphide in Blood Pressure Regulation. Physiol. Res. 2016, S273-S289. https://doi.org/10.33549/physiolres.933438.
(27) Filipovic, M. R.; Zivanovic, J.; Alvarez, B.; Banerjee, R. Chemical Biology of H₂S Signaling through Persulfidation. Chem. Rev. 2018, 118 (3), 1253-1337. https://doi.org/10.1021/acs.chemrev.7b00205.
(28) Zhao, W. The Vasorelaxant Effect of H₂S as a Novel Endogenous Gaseous KATP Channel Opener. EMBO J. 2001, 20 (21), 6008-6016. https://doi.org/10.1093/emboj/20.21.6008.
(29) Elrod, J. W.; Calvert, J. W.; Morrison, J.; Doeller, J. E.; Kraus, D. W.; Tao, L.; Jiao, X.; Scalia, R.; Kiss, L.; Szabo, C.; Kimura, H.; Chow, C.-W.; Lefer, D. J. Hydrogen Sulfide Attenuates Myocardial Ischemia-Reperfusion Injury by Preservation of Mitochondrial Function. Proc. Natl. Acad. Sci. 2007, 104 (39), 15560-15565. https://doi.org/10.1073/pnas.0705891104.
(30) Johansen, D.; Ytrehus, K.; Baxter, G. F. Exogenous Hydrogen Sulfide (H₂S) Protects against Regional Myocardial Ischemia-Reperfusion Injury: Evidence for a Role of KATP Channels. Basic Res. Cardiol. 2006, 101 (1), 53-60. https://doi.org/10.1007/s00395-005-0569-9.
(31) Bos, E. M.; van Goor, H.; Joles, J. A.; Whiteman, M.; Leuvenink, H. G. D. Hydrogen Sulfide: Physiological Properties and Therapeutic Potential in Ischaemia: Properties of H₂S in Ischaemia. Br. J. Pharmacol. 2015, 172 (6), 1479-1493. https://doi.org/10.1111/bph.12869.
(32) Muellner, M. K.; Schreier, S. M.; Laggner, H.; Hermann, M.; Esterbauer, H.; Exner, M.; Gmeiner, B. M. K.; Kapiotis, S. Hydrogen Sulfide Destroys Lipid Hydroperoxides in Oxidized LDL. Biochem. J. 2009, 420 (2), 277-281. https://doi.org/10.1042/BJ20082421.
(33) Panth, N.; Paudel, K. R.; Parajuli, K. Reactive Oxygen Species: A Key Hallmark of Cardiovascular Disease. Adv. Med. 2016, 2016, 1-12. https://doi.org/10.1155/2016/9152732.
(34) Sugamura, K.; Keaney, J. F. Reactive Oxygen Species in Cardiovascular Disease. Free Radic. Biol. Med. 2011, 51 (5), 978-992. https://doi.org/10.1016/j.freeradbiomed.2011.05.004.
(35) Peiró Cadahía, J.; Previtali, V.; Troelsen, N. S.; Clausen, M. H. Prodrug Strategies for Targeted Therapy Triggered by Reactive Oxygen Species. MedChemComm 2019, 10 (9), 1531-1549. https://doi.org/10.1039/C9MD00169G.
(36) Liou, G.-Y.; Storz, P. Reactive Oxygen Species in Cancer. Free Radic. Res. 2010, 44 (5), 479-496. https://doi.org/10.3109/10715761003667554.
(37) Wang, L.; Xie, S.; Ma, L.; Chen, Y.; Lu, W. 10-Boronic Acid Substituted Camptothecin as Prodrug of SN-38. Eur. J. Med. Chem. 2016, 116, 84-89. https://doi.org/10.1016/j.ejmech.2016.03.063.
(38) Kuang, Y.; Balakrishnan, K.; Gandhi, V.; Peng, X. Hydrogen Peroxide Inducible DNA Cross-Linking Agents: Targeted Anticancer Prodrugs. J. Am. Chem. Soc. 2011, 133 (48), 19278-19281. https://doi.org/10.1021/ja2073824.
(39) Ai, Y.; Obianom, O. N.; Kuser, M.; Li, Y.; Shu, Y.; Xue, F. Enhanced Tumor Selectivity of 5-Fluorouracil Using a Reactive Oxygen Species-Activated Prodrug Approach. ACS Med. Chem. Lett. 2019, 10 (1), 127-131. https://doi.org/10.1021/acsmedchemlett.8b00539.
(40) Skarbek, C.; Serra, S.; Maslah, H.; Rascol, E.; Labruère, R. Arylboronate Prodrugs of Doxorubicin as Promising Chemotherapy for Pancreatic Cancer. Bioorganic Chem. 2019, 91, 103158. https://doi.org/10.1016/j.bioorg.2019.103158.
(41) Steiger, A. K.; Pardue, S.; Kevil, C. G.; Pluth, M. D. Self-Immolative Thiocarbamates Provide Access to Triggered H₂S Donors and Analyte Replacement Fluorescent Probes. J. Am. Chem. Soc. 2016, 138 (23), 7256-7259. https://doi.org/10.1021/jacs.6b03780.
(42) Zhao, Y.; Pluth, M. D. Hydrogen Sulfide Donors Activated by Reactive Oxygen Species. Angew. Chem. Int. Ed. 2016, 55 (47), 14638-14642. https://doi.org/10.1002/anie.201608052.
(43) Lippert, A. R.; Van de Bittner, G. C.; Chang, C. J. Boronate Oxidation as a Bioorthogonal Reaction Approach for Studying the Chemistry of Hydrogen Peroxide in Living Systems. Acc. Chem. Res. 2011, 44 (9), 793-804. https://doi.org/10.1021/ar200126t.
(44) Greenwald, R. B.; Pendri, A.; Conover, C. D.; Zhao, H.; Choe, Y. H.; Martinez, A.; Shum, K.; Guan, S. Drug Delivery Systems Employing 1,4- or 1,6-Elimination: Poly(Ethylene Glycol) Prodrugs of Amine-Containing Compounds. J. Med. Chem. 1999, 42 (18), 3657-3667. https://doi.org/10.1021/jm990166e.
(45) Zhao, Y.; Henthorn, H. A.; Pluth, M. D. Kinetic Insights into Hydrogen Sulfide Delivery from Caged-Carbonyl Sulfide Isomeric Donor Platforms. J. Am. Chem. Soc. 2017, 139 (45), 16365-16376. https://doi.org/10.1021/jacs.7b09527.
(46) Hartle, M. D.; Pluth, M. D. A Practical Guide to Working with H₂S at the Interface of Chemistry and Biology. Chem. Soc. Rev. 2016, 45 (22), 6108-6117. https://doi.org/10.1039/C6CS00212A.
(47) Truong, D. H.; Mihajlovic, A.; Gunness, P.; Hindmarsh, W.; O'Brien, P. J. Prevention of Hydrogen Sulfide (H₂S)-Induced Mouse Lethality and Cytotoxicity by Hydroxocobalamin (Vitamin B12a). Toxicology 2007, 242 (1-3), 16-22. https://doi.org/10.1016/j.tox.2007.09.009.
(48) Mirzaei, S.; Zarrabi, A.; Hashemi, F.; Zabolian, A.; Saleki, H.; Azami, N.; Hamzehlou, S.; Farahani, M. V.; Hushmandi, K.; Ashrafizadeh, M.; Khan, H.; Kumar, A. P. Nrf2 Signaling Pathway in Chemoprotection and Doxorubicin Resistance: Potential Application in Drug Discovery. Antioxid. Basel Switz. 2021, 10 (3), 349. https://doi.org/10.3390/antiox10030349.
(49) Nordgren, K. K. S.; Wallace, K. B. Disruption of the Keap1/Nrf2-Antioxidant Response System After Chronic Doxorubicin Exposure In Vivo. Cardiovasc. Toxicol. 2020, 20 (6), 557-570. https://doi.org/10.1007/s12012-020-09581-7.
(50) Alzahrani, A. M.; Rajendran, P.; Veeraraghavan, V. P.; Hanieh, H. Cardiac Protective Effect of Kirenol against Doxorubicin-Induced Cardiac Hypertrophy in H9c2 Cells through Nrf2 Signaling via PI3K/AKT Pathways. Int. J. Mol. Sci. 2021, 22 (6), 3269. https://doi.org/10.3390/ijms22063269.
(51) Gu, J.; Huang, H.; Liu, C.; Jiang, B.; Li, M.; Liu, L.; Zhang, S. Pinocembrin Inhibited Cardiomyocyte Pyroptosis against Doxorubicin-Induced Cardiac Dysfunction via Regulating Nrf2/Sirt3 Signaling Pathway. Int. Immunopharmacol. 2021, 95, 107533. https://doi.org/10.1016/j.intimp.2021.107533.
(52) Yang, S.; Zhang, J. J.; Huang, X.-Y. Mouse Models for Tumor Metastasis. Methods Mol. Biol. Clifton NJ 2012, 928, 221-228. https://doi.org/10.1007/978-1-62703-008-3_17.
(53) Feliz-Mosquea, Y. R.; Christensen, A. A.; Wilson, A. S.; Westwood, B.; Varagic, J.; Meléndez, G. C.; Schwartz, A. L.; Chen, Q.-R.; Mathews Griner, L.; Guha, R.; Thomas, C. J.; Ferrer, M.; Merino, M. J.; Cook, K. L.; Roberts, D. D.; Soto-Pantoja, D. R. Combination of Anthracyclines and Anti-CD47 Therapy Inhibit Invasive Breast Cancer Growth While Preventing Cardiac Toxicity by Regulation of Autophagy. Breast Cancer Res. Treat. 2018, 172 (1), 69-82. https://doi.org/10.1007/s10549-018-4884-x.

The foregoing is illustrative of the present invention, and is not to be construed as limiting thereof. The invention is defined by the following claims, with equivalents of the claims to be included therein.

1. A compound of Formula I: 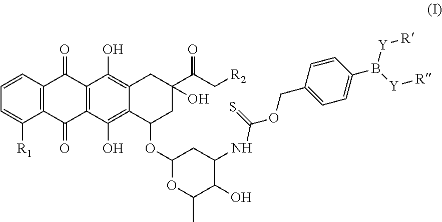
wherein:
R₁ is H or alkoxy;
R₂ is H or hydroxy
each Y is independently NRₐ or O; and
R′, R″, and Rₐ are each independently H or alkyl, optionally substituted, or R′ and R″ together with the Y atoms to which they are attached form a cyclic structure containing up to 5 carbon atoms, optionally substituted by one or more alkyl, heteroalkyl, aryl, heteroaryl, halogen, hydroxy, alkoxy, carbonyl and/or carboxy groups,
or a pharmaceutically acceptable salt thereof.

2. The compound of claim 1, wherein R′ and R″ together with the Y atoms to which they are attached form a boronate or diaminoboryl group.

3. The compound of claim 1, wherein said compound is a compound of Formula Ia: 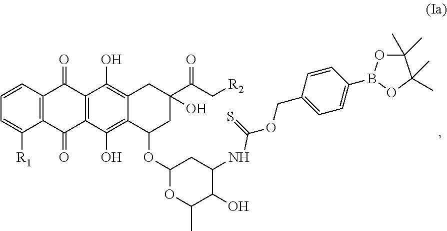
wherein R₁ is H or methoxy, and R₂ is H or hydroxy,
or a pharmaceutically acceptable salt thereof.

4. The compound of claim 1, wherein said compound is: 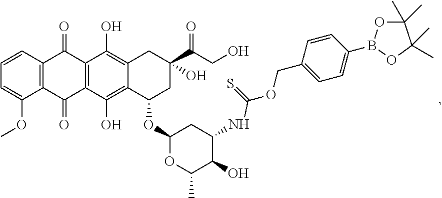 [SMILES: COc1cccc2c1C(=O)c1c(O)c3c(c(O)c1C2=O)C[C@@](O)(C(=O)CO)C[C@@H]3O[C@H]1C[C@H](NC(=S)OCc2ccc(B3OC(C)(C)C(C)(C)O3)cc2)[C@@H](O)[C@H](C)O1]
or a pharmaceutically acceptable salt thereof.

5. The compound of claim 1, wherein said compound is: 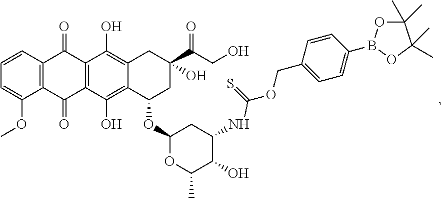 [SMILES: COc1cccc2c1C(=O)c1c(O)c3c(c(O)c1C2=O)C[C@@](O)(C(=O)CO)C[C@@H]3O[C@H]1C[C@H](NC(=S)OCc2ccc(B3OC(C)(C)C(C)(C)O3)cc2)[C@H](O)[C@H](C)O1]
or a pharmaceutically acceptable salt thereof.

6. The compound of claim 1, wherein said compound is: 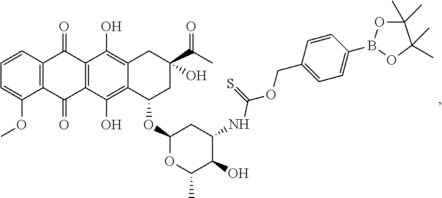 [SMILES: COc1cccc2c1C(=O)c1c(O)c3c(c(O)c1C2=O)C[C@@](O)(C(C)=O)C[C@@H]3O[C@H]1C[C@H](NC(=S)OCc2ccc(B3OC(C)(C)C(C)(C)O3)cc2)[C@@H](O)[C@H](C)O1]
or a pharmaceutically acceptable salt thereof.

7. The compound of claim 1, wherein said compound is: 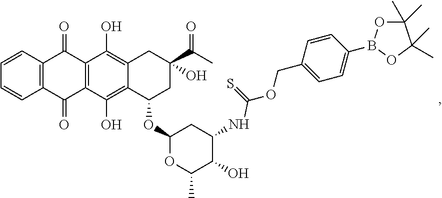 [SMILES: CC(=O)[C@]1(O)Cc2c(O)c3c(c(O)c2[C@@H](O[C@H]2C[C@H](NC(=S)OCc4ccc(B5OC(C)(C)C(C)(C)O5)cc4)[C@H](O)[C@H](C)O2)C1)C(=O)c1ccccc1C3=O]
or a pharmaceutically acceptable salt thereof.

8. A composition comprising the compound of claim 1 and a pharmaceutically acceptable carrier.

9. The composition of claim 8, wherein said compound is provided as a liposome formulation.

10. The composition of claim 8, wherein said compound is provided as a nanoparticle formulation.

11. A method of treating cancer in a subject in need thereof, comprising administering the compound of claim 1 to said subject in a treatment effective amount.

12. (canceled)

13. (canceled)

14. The method of claim 11, wherein the cancer comprises a solid tumor.

15. The method of claim 11, wherein the cancer is a soft tissue carcinoma.

16. The method of claim 11, wherein said cancer is breast cancer.

17. The method of claim 11, wherein the cancer is a leukemia.

18. The method of claim 11, wherein the cancer is a lymphoma.

19. The method of claim 11, wherein the cancer is bladder cancer.

20. The method of claim 11, wherein the cancer is kidney cancer.

21. The method of claim 11, wherein the cancer is ovarian cancer.

22. The method of claim 11, wherein said administering is carried out by parenteral administration.

23. The method of claim 22, wherein the parenteral administration is intravenous administration.
